(19) United States
(12) Patent Application Publication
(10) Pub. No.: US 2005/0196385 A1
(43) Pub. Date: Sep. 8, 2005

(54) Methods for producing gamma delta t cells

(75) Inventors: Francois Romagne, La Ciotat (FR); Catherine Laplace, Nantes (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Innate Pharama, Marseille (FR)

(21) Appl. No.: 10/505,252

(22) PCT Filed: Feb. 21, 2003
(86) PCT No.: PCT/FR03/00585

§ 371 Date: Aug. 19, 2004

(30) Foreign Application Priority Data
Feb. 22, 2002 (FR) 02/02305

(51) Int. Cl. A61K 45/00; A61K 31/663; C12N 5/08

(52) U.S. Cl. 424/93.7; 514/100; 514/102; 435/372

(57) ABSTRACT

The invention concerns methods for producing lymphocytic cells, as well as tools, reagents and kits useful for implementing same. More particularly, it concerns methods for preparing gamma delta T cells, adapted to industrial production of functional cells of pharmaceutical quality in large amounts. The invention also concerns methods for activating gamma delta T cells, devices adapted to said methods, as well as the resulting cell compositions and their human or animal gamma delta T cells, and can be used in pharmaceutics, therapeutics, experiments, cosmetics, industrial research among others.

The invention concerns methods for producing lymphocytic cells, as well as tools, reagents and kits useful for implementing same. More particularly, it concerns methods for preparing gamma delta T cells, adapted to industrial production of functional cells of pharmaceutical quality in large amounts. The invention also concerns methods for activating gamma delta T cells, devices adapted to said methods, as well as the resulting cell compositions and the uses thereof. The present application is applicable to the production of human or animal gamma delta T cells, and can be used in pharmaceutics, therapeutics, experiments, cosmetics, industrial research among others.

Gamma delta T cells normally account for 1 to 5% of peripheral blood lymphocytes in a healthy individual (human, monkey). They are involved in mounting a protective immune response, and it has been shown that they recognize their antigenic ligands by a direct interaction with antigen, without any presentation by MHC molecules of antigen-presenting cells. Gamma 9 delta 2 T cells (sometimes also called gamma 2 delta 2 T cells) are gamma delta T cells bearing TCR receptors with the variable domains Vγ9 and Vδ2. They form the majority of gamma delta T cells in human blood. When activated, gamma delta T cells exert potent, non-MHC restricted cytotoxic activity, especially efficient at killing various types of cells, particularly pathogenic cells. These may be cells infected by a virus (Poccia et al., J. Leukocyte Biology, 1997, 62: 1-5) or by other intracellular parasites, such as mycobacteria (Constant et al., Infection and Immunity, December 1995, vol. 63, no. 12: 4628-4633) or protozoa (Behr et al., Infection and Immunity, 1996, vol. 64, no. 8: 2892-2896). They may also be cancer cells (Poccia et al., J. Immunol., 159: 6009-6015; Fournie and Bonneville, Res. Immunol., 66^{th} Forum in Immunology, 147: 338-347). The possibility of modulating the activity of said cells in vitro, ex vivo or in vivo would therefore provide novel, effective therapeutic approaches in the treatment of various pathologies such as infectious diseases (particularly viral or parasitic), cancers, allergies, and even autoimmune and/or inflammatory disorders.

Different methods have been described in the prior art for producing such cells ex vivo or in vitro. For instance, application WO99/46365 discloses a method comprising a first culture of hemato-lymphoid cells in the presence of interleukin-12 and a CD2 ligand, followed by a second culture in the presence of a T cell mitogen compound and interleukin-2. Said method is complex, requires several steps of cell treatment and several metabolic activation pathways. Furthermore, it does not provide cell compositions sufficiently enriched in gamma delta T cells.

Applications WO00/12516 and WO00/12519 describe chemical compounds capable of activating gamma delta T cells. These applications propose the use of said compounds for activating an immune response in vivo, and additionally provide for the use of said compounds in methods for ex vivo or in vitro activation of gamma delta T cells. However, these applications do not provide for an industrial method by which to generate cell populations composed essentially of gamma delta T cells.

To envisage the use of gamma delta T cells for use in cell therapy, it is necessary to have a method for culturing and formulating cells that provides large amounts of cells highly purified for gamma delta T cells. The examples of LAK cell or T cell clones injections indicate that these treatments are only effective when large amounts of cells are injected (Bordignon, Haematologica, 1999, 84: 1110-1149 for review). Typically and on the basis of these examples, one must have at one's disposal a method by which to reproducibly obtain under pharmacopoeal conditions at least 100 million cells having a purity greater than 80%.

The present application now describes a novel method for producing gamma delta T cells. The method is adapted to industrial production of large amounts of cells, enables the production of functional gamma delta T cells of pharmaceutical quality. The method may be carried out directly on a cytapheresis, starting with large and heterogeneous quantities of cells, and allows very marked stimulation and expansion of gamma delta T cells, yielding compositions that can comprise more than 90% gamma delta T cells. Furthermore, the method according to the invention is simplified since it requires only one step or only one metabolic activation pathway. The method allows production of cell compositions adapted to different uses, particularly therapeutics.

A first object of the invention is based more particularly on a method for preparing a gamma delta T cell composition, comprising at least one step of culturing a biological preparation comprising at least 50 million mononuclear cells in the presence of a synthetic activator compound of gamma delta T cells at initiation of the culture, followed by a culture, typically from 10 to 25 days, in the presence of a cytokine. The resulting compositions advantageously have the following characteristics
they comprise more than 80% gamma delta T cells, and
they comprise more than 100 million viable and functional gamma delta T cells.

An advantageous characteristic of the inventive method is the possibility of starting with large amounts of unfractionated cells, to end up with preparations highly enriched in functional gamma delta T cells. Advantageously, the cells are maintained in culture at a cell density less than about 5. 106 cells/ml, preferably about 3.10⁶, more preferably about 2.10⁶ cells/ml. Indeed, the examples show that such density ensures efficient expansion of the cells.

Another object of the invention concerns a method for preparing a cell composition comprising functional gamma delta T cells, wherein it comprises
culturing a preparation of blood cells (typically cells from a cytapheresis) in the presence of a synthetic activator compound of gamma delta T cells and a cytokine selected in the group consisting of interleukin-2 and interleukin-15, said culture being carried out in conditions ensuring that cell density is maintained essentially below 5. 106 cells/ml, preferably at about 3.10⁶ cells/ml, and
recovering some or all of the resulting cells, said cells comprising functional gamma delta T cells.

Maintaining cell density may be accomplished in different ways, such as for example successive dilution(s), addition(s) of medium, change of culture device, among others.

Another object of the invention is a method for enriching blood cells in gamma delta T cells, comprising at least one step of culturing a biological preparation comprising at least 50 million blood mononuclear cells in the presence of a gamma delta T cell synthetic activator compound at initiation of the culture, followed by a culture, typically for 10 to 25 days, in the presence of a cytokine. The preparations obtained by said method can comprise more than 80%, or even more than 90%, of gamma delta T cells.

As indicated hereinbelow, the cells used are preferably human, may come from frozen biological samples, and are preferably cultured for a period of more than 10 days, preferably between 10 and 30 days.

Another object of the invention is a pharmaceutical composition, wherein it comprises a cell population composed of more than 80% functional gamma delta T cells and wherein it comprises more than 100 million gamma delta T cells. Preferably the composition also comprises a pharmaceutically acceptable agent or carrier and, more preferably, a stabilizing agent, such as human serum albumin. The cells are preferably autologous, that is to say, derived from a same biological preparation (or from a same donor). More preferably, they are obtained by a method such as described hereinabove.

Another object of the invention concerns a culture of blood cells in vitro or ex vivo, wherein it comprises at least 80% functional gamma delta T cells and more than 100 million gamma delta T cells.

The invention also relates to the use of a cell culture such as defined earlier for preparing a pharmaceutical composition for stimulating the immune defenses of a subject, more particularly for treating infectious, parasitic diseases or cancers.

The invention also concerns a method for treating a pathology that can be improved by increasing the activity of gamma delta T cells, particularly by enhancing the immune defenses of a subject, comprising administering to a subject an efficient amount of a pharmaceutical composition or cell composition such as defined hereinabove. The administration is preferably performed by injection, in particular by systemic injection (intravenous, intraperitoneal, intramuscular, intraarterial, subcutaneous and the like) or local (e.g., intratumoral or in a zone surrounding or irrigating a tumor). Repeated injections may be performed. The injected cells are preferably autologous (or syngeneic), that is to say, prepared from a biological preparation which comes from the patient himself (or from a twin). The method is useful in the treatment of various pathologies, such as cancers, infectious or parasitic diseases.

As indicated, the present invention can be used in pharmaceutics, therapeutics, experiments, cosmetics, industrial research among others. It is especially adapted to producing cell compositions for pharmaceutical use, particularly for increasing an immune response in a subject, for example for treating pathologies such as cancers and infectious or parasitic diseases.

Biological Preparation

The method according to the invention is advantageous in so far as it enables efficient production of gamma delta T cells from a biological preparation comprising large amounts of unfractionated blood cells. It may therefore be practiced directly on a sample of blood, plasma or serum, for example a cytapheresis. Typically, a blood mononuclear cell preparation, particularly from peripheral blood, is used. A peripheral blood cell preparation generally contains from 30 to 70% of T or B lymphocytes, from 5 to 15% of NK cells and from 1 to 5% of gamma delta T cells. Of course it is possible to treat the biological preparation before implementing the method according to the invention, for example to select certain subpopulations, or to deplete certain subpopulations. However, such pretreatment is not necessary to produce the functional gamma delta T cell compositions of the invention. Thus, the method is typically carried out directly on a blood cell sample collected from a subject, particularly a sample of total mononuclear cells (that is to say, unfractionated). Said sample may be obtained by conventional methods known to those skilled in the art and in widespread clinical use worldwide, such as cytapheresis or ficoll gradient on whole blood (PBMC). A preferred source of cells for practicing the invention is composed of total peripheral mononuclear cells such as obtained by cytapheresis. Thus, in a particular embodiment, the method according to the present invention comprises a first step of culturing a cytapheresis, or an aliquot of a cytapheresis, under the conditions described hereinabove. A cytapheresis typically provides more than 10⁹ mononuclear cells. From one cytapheresis, then, it is possible to prepare several aliquots, which can be treated separately by the method according to the invention. So, for a given patient, several batches of gamma delta T cells according to the invention can be produced, separately and at different times. This aliquoting makes it possible to perform quality and functional activity tests on the cells, and ensures a higher measure of safety of the compositions.

In this respect, the present application shows that functional gamma delta T cells can be produced from previously frozen mononuclear cell preparations. In fact, the results presented in the examples show that a cytapheresis can be frozen for long-term storage, and that the cells, after thawing, can be efficiently activated and expanded to produce functional gamma delta T cell compositions. This possibility of using previously frozen cells gives the invention a very important advantage, particularly in the context of preparing autologous cell banks.

A particular embodiment of the method according to the invention therefore comprises preparing gamma delta T cells from a previously frozen biological preparation (particularly of mononuclear cells).

A particular object of the invention also concerns a method for producing functional gamma delta T cells, comprising (i) culturing previously frozen blood mononuclear cells in the presence of a gamma delta T cell synthetic activator compound and a cytokine under conditions ensuring proliferation of gamma delta T cells and (ii) recovering or formulating the resulting gamma delta T cells. Preferably, the blood mononuclear cells are from a cytapheresis.

Another particular object of the invention concerns a method for producing functional gamma delta T cells, comprising (i) (obtaining and) freezing blood mononuclear cells from a subject, typically in the form of aliquots containing about 10⁷ to 5.10⁹ cells per ml, (ii) thawing the cells or the individual aliquots and culturing them in the presence of a gamma delta T cell synthetic activator compound and a cytokine under conditions ensuring proliferation of gamma delta T cells, and (iii) recovering or formulating the resulting gamma delta T cells. Preferably, the blood mononuclear cells are from a cytapheresis.

The cells may be frozen by various methods. A preferred method makes use of a stabilizing agent such as DMSO (dimethylsulfoxide) and/or gylcerol. Such agent stabilizes cell membranes and allows efficient freezing of the cells, in terms of cell viability after thawing. Other techniques or media may be used, based on gelatins, polymers, proteins and the like. A particularly suited medium is a 90/10 (V/V) solution of serum and DMSO, where the serum used also serves to promote cell proliferation. The percentage of DMSO may range from 5 to 15% by volume of the solution. The serum may be replaced by a 4% solution of human serum albumin, for example Albumine-LFB 4% (marketing authorization number 558632-9). The percentage of human serum albumin may however be higher, for instance up to 20%. Typically, the cells are suspended in a suitable freezing medium, such as defined hereinabove, then placed in a freezing atmosphere, such as liquid nitrogen vapors, for example. Freezing is advantageously carried out in suitable tubes or bags, under sterile conditions, in the form of aliquots of a same blood cell preparation. The cells so frozen may be stored for very long periods, thereby allowing the production of gamma delta T cells over long intervals, without the need for repeated sampling from a subject.

An important characteristic of the method according to the invention is the quantity of biological material treated. For instance, the method preferably uses a biological preparation comprising more than 50.10⁶ mononuclear cells, typically between 50 and 1000 million cells, for example about 50, 100, 200 or 300 million cells. In a typical method, the biological preparation comprises more than 100 million cells. It is understood that larger amounts may be used. In so far as a typical biological preparation comprises at the outset less than 10% gamma delta T cells, usually less than 5% gamma delta T cells, a biological preparation of 100 million cells typically contains from 1 to 5 million gamma delta T cells. From such preparations, the method according to the invention yields compositions comprising 108 or more functional gamma delta T cells. Moreover, while the starting preparations contain only about 1 to 5% of gamma delta T cells, the compositions obtained by the method according to the invention are composed of more than 80%, even more than 90%, of gamma delta T cells. The method according to the invention is therefore particularly efficient and adapted to the production of cells in large amounts and of pharmaceutical quality.

Synthetic Activator Compound of Gamma Delta T Cells

An advantageous aspect of the method according to the invention is the use of a synthetic activator compound of gamma delta T cells. Hence, the invention shows that an efficient and oriented activation and expansion of gamma delta T cells may be achieved by a single metabolic activation by means of a synthetic compound.

The term synthetic activator compound indicates that the invention makes use of an artificially produced molecule, capable of activating gamma delta T cells. It is typically a ligand (e.g., a chemical molecule) capable of binding to the T-cell receptor of gamma delta T cells. The activator compound may be of various nature, such as a peptide, lipid, chemical among others. It may be a endogenous ligand purified or produced by chemical synthesis, or a fragment or derivative of said ligand, or an antibody having the same antigenic specificity. Preferably it is a synthetic chemical compound, capable of selectively binding to the TCR receptor and activating gamma delta T cells. Selective binding indicates that the compound interacts with a higher affinity at the TCR of gamma delta T cells than at other membrane receptors, and therefore leads to selective or oriented activation of gamma delta T cell proliferation and activity.

Different synthetic activator compounds may be used, such as the phosphohalohydrins (PHD) described in application WO00/12516, the phosphoepoxides (PED) described in application WO00/12519, or the bisphosphonate compounds such as described by Kunzmann et al. (Blood, 2000, 96: 384).

Specific synthetic activator compounds which may be used advantageously to implement the invention are the phosphohalohydrins and phosphoepoxides represented of formulas (I) and (II) below, respectively: 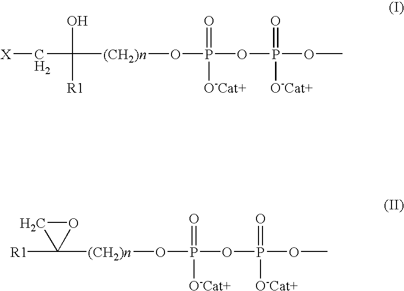
in which X is a halogen atom (preferably selected in the group consisting of an iodine, bromine or chlorine atom), R1 is a methyl or ethyl group, Cat+ represents a mineral or organic cation(s) (including the proton), which are the same or different, and n is an integer comprised between 2 and 20. Said compounds may be produced by different chemical methods known to those skilled in the art, and in particular the methods described in applications WO00/12516 and WO00/12519. Particular compounds are the di- or tri-phosphate compounds represented by formula (I) or (II) hereinabove.

In a preferred embodiment, a PED or PHD compound is used. Specific compounds are the following:
3-(bromomethyl)-3-butanol-1-yl-diphosphate (BrHPP)
3-(iodomethyl)-3-butanol-1-yl-diphosphate (IHPP)
3-(chloromethyl)-3-butanol-1-yl-diphosphate (ClHPP)
3-(bromomethyl)-3-butanol-1-yl-triphosphate (BrHPPP)
3-(iodomethyl)-3-butanol-1-yl-triphosphate (IHPPP)
α,γ-di-[3-(bromomethyl)-3-butanol-1-yl]-triphosphate (diBrHTP)
α,γ-di-[3-(iodomethyl)-3-butanol-1-yl]-triphosphate (diIHTP)
3,4,-epoxy-3-methyl-1-butyl-diphosphate (Epox-PP)
3,4,-epoxy-3-methyl-1-butyl-triphosphate (Epox-PPP)
α,γ-di-3,4,-epoxy-3-methyl-1-butyl-triphosphate (di-Epox-TP)

In another particular embodiment, the aminobiphosphonate compounds are used, such as for example 1-hydroxy-3-(methylpentylamino)propylidene-biphosphonic acid.

In another variant, the synthetic activator is (E)-4-hydroxy-3-methyl-but-2-enyl pyrophosphate, such as described by Hintz et al. (FEBS Lett., Dec. 7 2001, 509(2): 317-22)

Although less efficient, other compounds useful for practicing the invention are the phosphoantigens described in application WO95/20673 or isopentenyl pyrophosphate (IPP) (U.S. Pat. No. 5,639,653).

The dose of activator compound may be adapted by those skilled in the art according to the quantity of cells and the nature of the compound used. In general, the compound is used at the initiation of the culture at a concentration less than or equal to about 10 μM. An important advantage of the method according to the invention is the fact that only one selective metabolic activation is necessary, at the start of culture. Thus, once the culture is initiated, there is no longer any need to add more synthetic activator compound to the medium.

Cytokine

The method according to the invention makes use of a cytokine (alone or possibly combined or associated with other biologically active agents), in particular an interleukin. Advantageously this is interleukin-2 or interleukin-15. In fact, the present application shows that said interleukins, which use the same receptor, allow efficient production of gamma delta T cells, in the conditions described hereinabove.

The interleukin used may be of human or animal origin, preferably of human origin. It may be a wild-type protein or any biologically active fragment or variant, that is to say, capable of binding to its receptor and inducing activation of gamma delta T cells in the conditions of the method according to the invention.

In particular, the term “variant” denotes all natural variants, resulting for example from polymorphism(s), splicing(s), mutations(s), etc. Said natural variants may therefore comprise one or more mutations or substitutions, a deletion of one or several residues, etc. relative to the wild-type sequence. The term variant also encompasses polypeptides originating from another species, for example rodents, bovines, etc. Advantageously, however, a human cytokine is used. The term “variant” also includes any synthetic variant of a cytokine, and particularly any polypeptide comprising one or several mutations, deletions, substitutions and/or additions of one or several amino acids relative to the wild-type sequence. Preferred variants advantageously show at least 75% primary sequence identity to the wild-type cytokine, preferably at least 80%, more preferably at least 85%. Even more preferably, the preferred variants show at least 90% primary sequence identity to the wild-type cytokine. The degree of identity may be determined by different methods and by means of software known to those skilled in the art, such as by the CLUSTAL method for example.

As indicated, it is also possible in the scope of the invention to use any cytokine fragment conserving the biological activity defined hereinabove. Said fragments preferably contain at least one region or one functional domain of the cytokine, such as for example a catalytic domain, a receptor binding site, a secondary structure (loop, sheet, etc.), a consensus site, etc. To implement the invention, the fragments used advantageously conserve the property of interleukin-2 or interleukin-5 to bind to the membrane receptor and stimulate the development of gamma delta T cells.

The cytokines used may additionally contain heterologous residues added to the wild-type amino acid sequence, such as amino acids, lipids, sugars and the like. They may also be chemical, enzymatic, radiolabelled group(s) and the like. In particular, the heterologous moiety may be a stabilizing agent, an agent facilitating penetration of the polypeptide into cells or improving its affinity, etc.

The cytokines may be in soluble, purified form, fusioned or complexed with another molecule, such as for example a peptide, polypeptide or biologically active protein. The cytokines may be prepared by any biological, genetic, chemical or enzymatic method known to those skilled in the art, and in particular by expression of a corresponding nucleic acid in a suitable host cell. Cytokines such as IL-2 and IL-15 may also be obtained commercially. Preferably, a human recombinant cytokine is used, typically a human recombinant interleukin-2 or a human recombinant interleukin-15.

The cytokine doses used in the method according to the invention may vary according to the nature of the starting cells. Moreover, the cytokine concentration may be modified during culture. Typically, the cytokines are used at concentrations comprised between 100 and 500 U/ml, typically between approximately 150 and 500 U/ml. As the method progresses, the cytokine concentration may be adjusted, for example by adding culture medium. In a preferred manner, cytokine doses comprised between 150 and 400 U/ml are used. In a particular embodiment, it is possible to start the culture in the presence of a first cytokine dose, then to continue it in the presence of a second dose, which is higher than the first, so as to increase cell proliferation. Thus, a particular object of the invention is based on a method for preparing a gamma delta T cell composition from a mononuclear cell sample, comprising at least:
a first culture step of mononuclear cells in the presence of a gamma delta T cell synthetic activator compound and a cytokine, said cytokine being present at a first effective dose, and
a second culture step of said cells in the presence of a second effective dose of said cytokine, said second effective dose being higher than said first effective dose.

As a matter of fact, the invention shows that the use of a synthetic activator compound promotes the expression of high affinity receptors for the cytokine IL2 at the surface of gamma delta T cells, and that low doses of IL-2 are sufficient to allow specific proliferation of gamma delta T cells, said low dose not promoting the growth of cells bearing lower affinity receptors. However, the high affinity receptor disappears after 7 to 10 days of culture and is replaced by a lower affinity receptor. The cells must then be cultured in the presence of a second, higher dose of cytokine, so as to improve the performance of the method and the proliferation of functional gamma delta T cells. In this embodiment, the first cytokine dose is preferably a dose less than or equal to about 300 U/ml, preferably on the order of about 150 U/ml, and the second cytokine dose is preferably a dose greater than about 300 U/ml, preferably about 350 U/ml, typically about 400 U/ml.

In another embodiment, the cytokine concentration is held essentially constant during the method, for example by adding fresh medium containing the cytokine at different times. Preferably, in this embodiment the cytokine concentration is held between 250 and 500 U/ml, for example between 300 and 450 U/mi.

Culture

The method according to the invention comprises culturing cells in the presence of an activator compound (at the initiation of culture) and a cytokine, for a time period and in conditions allowing selective activation and expansion of gamma delta T cells.

The cells may be cultured in different media and devices adapted to the culture of human cells, particularly blood cells. These may be defined, supplemented media and the like. Useful media are exemplified in particular by the commercially available media RPMI, Prolifix S3, S6, Ampicell X3 (Bio Media), X-VIVO-10 and 15 (Biowhittaker), AIM V (Invitrogen), Medium I and II (Sigma), StemSpan H200 (Stem cell), CeliGro SCGM (CeliGenix), etc. Said media may be supplemented with antibiotics, human or animal serum, preferably authorized for use in cell culture for therapeutic use, amino acids and/or vitamins, etc. A preferred medium is RPMI medium, preferably supplemented with fetal calf serum. An especially preferred medium is an irradiated calf serum, holding regulatory authorization for use in therapeutic cell culture. This type of serum is commercially available from several suppliers. The cultures may be carried out in different devices, such as plates, bags, flasks, bottles, tubes, ampoules, bioreactors, etc. The cultures are advantageously carried out in sterile devices which can be sealed. It is not necessary to shake the cultures. Gas-permeable bags are especially suited. Depending on the conduct of the method, the devices may be changed during culture, particularly to dilute to the cells and promote their expansion. However, such change is not mandatory, and large-volume devices may be used from the outset of the method and maintained throughout.

Typically, when bags are used, the biological preparation or cells are contained in a volume of medium such that the initial cell density is comprised between 0.2 and 3.10⁶ cells/ml. In fact, the applicants have shown that maintaining a cell density comprised between 0.2 and 3.10⁶ cells/ml, more preferably close to 2.10⁶ cells/ml, strongly promotes the expansion of gamma delta T cells. Higher concentrations of cells could probably be obtained by using bioreactors.

Cell density may be maintained in different ways, such as for example by successive dilution(s), addition(s) of medium, change of culture device and the like. Of course, cell density cannot be held constant during the method, since the cells are continually dividing. Advantageously, then, density is controlled or adjusted at different times, so as to maintain as closely as possible a density comprised between 0.5 and 3.10⁶ cells/ml.

The method may be conducted for variable time periods and/or in several cycles. In general, the duration of the method is more than approximately 10 days, typically comprised between about 10 and 30 days or between about 10 and 25 days. Different variants may be envisioned. For instance, it is possible in a first phase to carry out the culture in the presence of the activator compound alone and in the absence of cytokine. Said first phase may last from 1 hour to 72 hours for example, typically less than 48 hours. Said phase is intended to stimulate the gamma delta T cells and induce some expression of high affinity cytokine receptors by these cells. At the end of this first phase, the culture is continued in a medium containing the cytokine, but without it being necessary to re-add the activator compound. Typically, at the end of this phase, fresh medium containing the cytokine, but without activator compound, is added to the cells. The culture is then continued for a period of more than approximately 10 days, typically between 10 and 25 days. As indicated, cell density is preferably tested and/or adjusted during culture, and the cytokine dose used may be maintained or modified.

According to another variant, it is possible to initiate the culture in the presence of the activator compound and the cytokine, and to continue it for a period typically comprised between 10 and 30 days, while controlling cell density and cytokine concentration. Thus, as a function of cell density, fresh medium containing the cytokine (but typically not the activator compound) is added to the cells. Moreover, as noted earlier, the cells may be separated or transferred during the procedure into larger volume devices, where necessary.

As indicated, the method according to the invention provides cell compositions which advantageously have the following characteristics:
they comprise more than 80% gamma delta T cells, advantageously more than 85%, even more than 90%, and
they comprise more than 100 million viable and functional gamma delta T cells.

To reproducibly obtain such characteristics in the majority of donors, it is necessary to start the culture with a large number of cells, roughly 50 million PMBC obtained for example from cytapheresis.

The method is simple, rapid, requires only one metabolic activation, and involves a very small number of manipulations of the cells. Furthermore it may be implemented using previously frozen cells. Said method is therefore particularly advantageous for pharmaceutical use of gamma delta T cells.

Uses/Formulation

The cells produced may be used extemporaneously or treated in view of storage. In general, the cells are packaged in a medium containing a stabilizing agent, such as a polymer or a neutral protein in particular. Human serum albumin (HAS) is advantageously used, and parenteral grade preparations are commercially available. The results presented herein show that the cells may be formulated in a human serum albumin solution at 4° C., in view of their injection. In this respect, a particular object of the invention is a composition comprising gamma delta T cells and human serum albumin, typically from 2 to 10%, advantageously about 4%.

Another object of the invention is a pharmaceutical composition, wherein it comprises a cell population composed of more than 80% functional gamma delta T cells and comprising more than 100 million gamma delta T cells. Preferably, the composition comprises more than 85% functional gamma delta T cells, even more than 90%. In general, the composition additionally comprises a pharmaceutically acceptable agent or carrier and, more preferably, a stabilizing agent, such as human serum albumin. More preferably, the cells are obtained or can be obtained by a method such as described hereinabove.

Another object of the invention concerns a method for preparing a pharmaceutical composition based on gamma delta T cells, the method comprising:
culturing cells according to the method described in the present application,
recovering some or all of the obtained cells, said cells comprising functional gamma delta T cells, and
formulating the cells in a pharmaceutically acceptable agent or carrier.

Another object of the invention concerns a in vitro or ex vivo blood cell culture, wherein it comprises at least 80% functional gamma delta T cells.

The invention also relates to the use of a cell culture such as defined hereinabove for preparing a pharmaceutical composition for stimulating the immune defenses of a subject, more particularly for treating infectious, parasitic diseases, cancers, autoimmune or inflammatory diseases.

The invention also concerns a method for treating a cancer or infectious or parasitic disease, comprising administering to a subject an efficient amount of a pharmaceutical composition or cell composition such as defined hereinabove.

The term treatment denotes a reduction or disappearance of symptoms, causes or sites of disease, a regression or slowing of disease progression, for example of tumor growth, an improvement in the condition of patients, a reduction in viral or parasite load, an alleviation of pain or suffering, an increase in survival, and the like.

The term efficient amount more particularly refers to an amount that is effective at stimulating a patient's immune response against the cancerous or infected cells. The cells are administered at doses typically comprised between 10⁶ and 10¹⁰ cells per dose, although different amounts may be used. It is understood that the amount of cells used may be adjusted by the practitioner according to the pathology and the clinical protocol (particularly the number and site of injections).

Administration is preferably by injection, in particular by systemic injection (intravenous, intraperitoneal, intramuscular, intraarterial, subcutaneous, etc.) or local injection (e.g., intratumoral or in a zone surrounding or irrigating the tumor). Repeated injections may be given. The injected cells are preferably autologous (or syngeneic), that is to say, they are prepared from a biological preparation from the patient himself (or from a twin). Allogeneic compositions may be envisioned.

In a typical embodiment, repeated injections are given, with dose escalation, each dose level itself possibly comprising several injections (typically from one to four) at time intervals ranging from one to six weeks for example. The initial dose is typically greater than 100 million cells, for example comprised between 100 million and 5 billion, and dose escalation up to 10 billion cells may be performed. A particular clinical protocol involves a dose escalation (each dose level comprising three successive injections at three week intervals) starting at 1 billion, then 4 billion, then 8 and then 12 billion cells. Furthermore, as the proliferation and survival of gamma 9 delta 2 cells depends on the activity of cytokines and, preferably, interleukin 2, cotherapy is advantageously performed. For instance, in a preferred embodiment, the cells obtained by the method according to the present invention are injected in cotherapy with a cytokine, particularly IL-2. A preferred dosing regimen consists in daily subcutaneous injections for about 7 days of about 1 million units of cytokine per square meter of body surface area.

A particular object of the invention is therefore based on a composition comprising cells such as defined hereinabove and a cytokine, preferably IL-2 or IL-15, more preferably IL-2, in view of their use simultaneously, separately or spread out over time. A further object of the invention is based on a method of treatment comprising administering to a subject a cell composition such as defined hereinabove and a cytokine, preferably IL-2, the cells and the cytokine being administered simultaneously, separately or spread out over time. Furthermore, the gamma delta T cells may be genetically modified, prior to administering them, for example to express a stimulation factor, growth factor, cytokine, toxin, among others.

The invention is useful (alone or in association with other therapies) for treating different pathologies which can be improved by increasing the activity of gamma delta T cells (and particularly those involving cells susceptible to the cytolytic activity of gamma delta T cells). Thus, most renal carcinoma tumor cell lines are killed efficiently in vitro by gamma 9 delta 2 cells obtained by the method according to the invention. Different histologic types of cancer may also be treated, on which the gamma delta cells exert cytolytic activity: myeloma, bladder cancer, melanoma, astrocytoma, neuroblastoma. This list is not exhaustive, and other types of cancers susceptible to gamma delta cell lysis may also be treated (lung, liver, head and neck, colorectal cancers, etc.). In the case of infectious diseases, gamma delta T cells have been shown to lyse many intracellular bacteria and mycobacteria. For instance, the activity of gamma 9 delta 2 cells against tuberculosis-infected cells or plague-infected cells is well known. Said cells also act against other infectious diseases such as tularemia. Antiviral activity has also been demonstrated against cells infected by HIV, influenza, Sendai, coxsackie, vaccinia virus, vesicular stomatitis virus (VSV), and herpes simplex virus-1 (HSV-1) (Sciammas et al., TcR gamma delta and viruses, Microbes Infect. 1999, 1: 203).

Other aspects and advantages of the present application will become apparent in the following examples, which are given for purposes of illustration and not by way of limitation.

### EXAMPLES

### Example 1

### Expansion of Gamma 9 Delta 2 T Cells Starting from More than 50 Million Unfractionated PMBC Cells so as to Obtain, After 10-20 Days of Culture, Greater than 80% Purity in Gamma 9 Delta 2 T Cells and More than 100 Million Gamma 9 Delta 2 T Cells

IA—Materials

Blood Samples

Whole blood was drawn into 6-mL tubes (on ACD: Acid Citrate Dextrose) from each of three healthy donors and stored at room temperature.

The blood was treated approximately 18 hours after sampling.

Cytapheresis Bags

A cytapheresis bag (½ body mass) was collected from healthy donors and stored at room temperature.

MNC (mononuclear cells) were treated approximately 18 hours after collection.

Culture Media

Different culture media, synthetic or otherwise, were used. Cells were cultured in RPMI medium (Sigma, ref. R0883), possibly supplemented with L-glutamine (0.3 g/l final) immediately before use.

Different synthetic media were also tested, as shown in Table 1.

In some cases, the media were supplemented with human or animal serum. In this respect, irradiated fetal calf serum was used (“Fetal Clone-I” irradiated with 25 kGy, from Hyclone (ref. SH 30080.03 IR)), as well as human serum.

The human serum used in these studies was from pooled sera of healthy donors prepared at the transfusion center in Nantes. This therapeutic grade serum (authorized by the French regulatory agency) is used in cell therapy protocols involving injection of classical alpha beta T cells.

Compounds and Reagents

The human recombinant interleukin-2 used was Proleukin (Aldesleukin) containing 18 million IU (Chiron BV, ref. FRC01A) stored in aliquots at a concentration of 360,000 IU/ml in RPMI/10% HS medium at −20° C. Ficoll (“Lymphocyte separation medium”) was used at a density 1.077±0.001 (Sigma, ref. 913353). The human serum albumin was albumin-LFB 4%, which holds the marketing authorization number 558632-9. DMSO and saline solution were from Braun Medical.

Culture Devices

The culture devices used are listed in Table 2.

Antibodies

The antibodies used are listed in Table 3.

IB—Methods

Isolation of Lymphocytes from Whole Blood+Ficoll

This procedure is commonly used in cell biology laboratories. Briefly, whole blood was subjected to Ficoll treatment and PBMC were then recovered on a Ficoll gradient. The Ficoll was rinsed, and cells were counted on a “Coulter Multisizer II” (on three different samples for a given condition and for a given donor). PBMC were frozen in a freezing solution containing 10% DMSO (in 4% human serum albumin or in FCS).

Isolation of Lymphocytes from MNC (Cytapheresis)

This procedure comprises a first phase of “platelet depletion” of the sample, which was performed on each cytapheresis bag as described below:

The contents of the cytapheresis bag were transferred to 50 ml tubes to which 2 volumes of RPMI medium were added. The tubes were centrifuged at 200 g, and the supernatant was discarded. The pellets were combined (pooled) and suspended in RPMI medium (qsp 50 ml). The cells were counted and re-centrifuged at approximately 400 g (at 20° C). The supernatant was again discarded, and the pellet was suspended in fetal calf serum so as to have a final cell concentration of approximately 500 million cells/ml. The cells were counted and the cell concentration was adjusted to approximately 300 million cells/ml with fetal calf serum. The cell suspensions were usually placed on ice (4° C.). MNC can be frozen in freezing solution containing 5 to 15% DMSO (in 4% human serum albumin or in FCS), or directly cultured.

Freezing of Cells

To optimize the freezing parameters, cells suspended in 4% human serum albumin or FCS were diluted volume to volume in refrigerated freezing solution (20% DMSO and 4% human serum albumin or FCS). The tube containing the cell suspension was shaken throughout the operation and advantageously left in a refrigerated container or on shaved ice. The homogenized mixture was distributed into 1.8 ml cryotubes (1 ml per tube) which were placed in a freezer box and stored at −80° C. The cryotubes were then transferred and stored in liquid nitrogen (at least 4 hours later).

MNC and PBMC were quick-thawed (by immersion in a water bath at 37° C.), then transferred into 15 ml tubes containing 12 ml of RPMI medium. The cells were washed with RPMI/10% FCS to eliminate the DMSO. The cells were counted on a Coulter counter (on three different samples for a given condition and for a given donor).

Initiation of Culture in Flasks and Bass (on the Day of Isolation)

The number of MNC seeded into the different containers (or culture devices) was selected in proportion to the ratio “number of lymphocytes/area per well” used for culture on 24-well plates, equivalent to about 1.10⁶ cells/1.9 cm² (see Table 4).

Mononuclear cells from each donor were cultured in containers in a same starting volume and number of cells, i.e., 100 million cells per container, in 50 ml of RPMI/10% FCS/3 μM BrHPP, 120 IU/ml IL-2 (equivalent to an initial cell concentration of 2 million/ml). The same medium containing 360 IU/ml IL2 was added during the culture as indicated for each manipulation.

Initiation of Culture in 24-Well Plates (After Thawing)

PBMC and MNC were cultured in 24-well plates at a rate of 1 million cells per well in 1.5 ml of RPMI/10% FCS/3 μM BrHPP/120 IU/ml IL-2 (equivalent to a cell concentration of 0.6 million/ml)

Culture Conditions

Cells were maintained in culture at 37° C. in a humidified, 5% CO₂ atmosphere in RPMI/10% FCS/360 IU/ml IL-2. The first medium change took place by adding medium at day 4, and then every 3 days. Thus, the concentration of IL2 increased during culture.

Cells grown in 24-well plates were transferred to 25 cm² flasks positioned vertically, when the cell density exceeded 3.10⁶ cells/ml.

For cells cultured in flasks or bags, cell density was held at 2.10⁶ cells/ml by addition of culture medium: Vmax=150 ml. Bearing in mind that the same culture container was used throughout the culture, once the maximum volume was reached, it was necessary to remove some of the cells so as to maintain cell density at 2.10⁶ cells/ml (and addition of fresh medium).

Counting, Phenotyping (Flow Cytometer Analysis)

Cells were counted and phenotyped several times during the three weeks of culture, in particular on days D10, D15, D20. Cell counts and phenotyping were carried out as follows:
Coulter cell count of all viable cells
CD56/CD3 double labelling
V62/CD3/CD69 triple labelling
Isotypic controls: IgG1k-FITC/R-PE/cyC
Data acquisition by flow cytometry (FACScan, Becton Dickinson)

Cell counts could be performed at other time points, in case of rapid cell expansion, so as to complete with fresh medium.

Functional Analysis of the Cells

Different tests were carried out on the cells obtained to evaluate their functional activity. In particular, said tests concerned the cytotoxic activity of the cells and their production of TNF.
Cytotoxicity test. For this test, the target cells were isotopically labelled with ⁵¹Cr (10 μl of ⁵¹Cr/1 million target cells in 24-well plates), then incubated for 1 hour at 37° C. The cells were distributed (in duplicate) at a rate of 3000 cells per well in RPMI/10% FCS (50 μl), and spontaneous and maximum release of ⁵¹Cr were determined. Effector cells (gamma delta T cells of the invention) were then added (50 μl in RPMI/10% FCS) to each target, at the following effector/target (E/T) ratios: 30/1, 3/1, 0.3/1, and incubated for 3 to 4 hours at 37° C. Cytotoxic activity (lysis of target cells) was determined by measuring the released radioactivity on 25 μl of supernatant in a β plate counter.
TNF release test. Cells were washed twice in RPMI then cultured in 96-well plates in RPMI/10% FCS in the presence of 3 μM BrHPP for 24 hours. TNF was assayed in the supernatant using the Beckman Coulter Kit Immunotech, ref. IM 11121.
   IC—Results
   Choice of Medium

Media supplemented with human sera were considered most favorable for culturing human lymphocytes and particularly gamma 9 delta 2 T cells, mainly due to the fact that serum growth factors are often species-specific. However, such media are very difficult to prepare and to use in the clinic, due to the biological risk and availability of large quantities of human sera.

An attempt was therefore made to culture the cells in media that were easier to prepare. These were small-scale experiments conducted in 24-well plates using whole blood from three different donors with an initial activation by EpoxPP (see Materials and Methods for activation conditions). The concentration and number of gamma delta T cells were followed over approximately 30 days of culture by cell counting and flow cytometry. The results of a comparative growth test in RPMI medium supplemented either with human serum or FCS, and in two synthetic media (X-VIVO 10 and 15) on three healthy donors, are given in Table 5.

In a surprising manner, the best medium for growth of gamma 9 delta 2 T cells was RPMI medium supplemented with FCS. Medium supplemented with human serum also afforded very significant growth of the gamma 9 delta 2 T cells, although to a lesser extent and with variations between different donors. Moreover, the purity and cell counts deteriorated over time in comparison with the FCS-supplemented medium. The synthetic media tested (serum-free) gave less cell growth, even though these are the best synthetic growth media for gamma 9 delta 2 T cells (data not shown).

In conclusion, gamma delta T cells have a very high long-term growth potential, in a favorable medium. FCS was chosen for subsequent studies because it gave very interesting and reproducible results from one batch to another (data not shown). It is also available in irradiated batches authorized for therapeutic uses. It is likely that other media might also reveal the high growth potential of gamma delta T cells, such as media with less serum, combinations of the best synthetic media with small amounts of serum, or combinations of synthetic media.

PBL Versus Cytapheresis

As the objective is to produce large quantities of gamma 9 delta 2 T cells at the end of the culture, it was of interest to start with a source containing a large number of cells, that could eventually be frozen, to be able to have cell banks. One possible source is represented by the mononuclear cells obtained by cytapheresis. However, this procedure can alter the cells and impede satisfactory cell growth. As cytapheresis samples often contain numerous red blood cells, the MNC were tested for proliferation, either just after platelet depletion or after platelet depletion and Ficoll treatment (see Materials and Methods). Thus, cells from a cytapheresis were tested to see if they could exhibit satisfactory growth. This test was first carried out on a small scale (24-well plates, see Materials and Methods), and Table 6 shows the results from three different donors.

Surprisingly, it was seen that cytapheresis cells also have a very high growth potential, though less than that of PMBC from whole blood. Furthermore, Ficoll-treated MNC showed lower growth than untreated MNC. In spite of the lower growth, and considering the quantities of cells needed at the start to yield large numbers of cells, a larger scale culture was done starting with fresh MNC cells not subjected to Ficoll.

Thus, a proliferation test was carried out on fresh MNC from healthy donors (D100, D119, D127). Different culture vessels were tested (see Materials and Methods). The culture was initiated with 100 million MNC cells at a concentration of 2 million cells/ml (total starting volume 50 ml). Cells were stimulated with 3 μM BrHPP. Fifty milliliters of fresh medium (containing 350 U/ml IL2) were added at days 4 and 7. Starting from day 10, the cells were analyzed and counted, and adjusted to 2 million cells/ml. They were then diluted every three days to maintain the cell density at 2 million cells/ml. The results are shown in Table 7.

It can be seen that, in the conditions of the invention, values of 100 million gamma 9 delta 2 T cells with over 80% purity were achieved starting from D10 for certain donors with certain culture devices (D100 and D119 in Nexell bags for example). These results demonstrate the efficiency of the methods according to the invention in producing functional gamma delta T cells of pharmaceutical quality.

### Example II

### Study of the Maintenance Cell Concentration After Day 10

IIA—Expansion of Gamma 9 Delta 2 T Cells

Another experiment, based on the previous method, was carried out with MNC from three new donors (D623, D762, D711), using the same materials and methods as in example I unless otherwise indicated. The culture initiation conditions were identical. Fifty milliliters of medium were added at days 4 and 7. The cells were analyzed and counted at day 10. The culture was performed in triplicate until day 10 (3 identical cultures per donor). The cell density was then adjusted to 3 concentrations (0.2, 0.5 and 1 million cells per ml, one triplicate being used for each concentration), so as to study the effect of the cell density parameter. Cultures were then analyzed every three days, and the density was adjusted to that at day 10 when it exceeded 2 million cells per ml. The results are given in Table 8.

It can be seen that cell yield and purity were much higher after day 10 than in example I. Cell density is therefore an important factor in performing the culture after this time point. In this manner it was discovered that the growth potential of gamma delta T cells is excellent. Starting with 1 to 4 million gamma T delta cells, 11 to 13 billion cells with purity greater than 90% were obtained at day 21. Importantly, the number of cells obtained does not appear to depend on the initial number of gamma delta T cells.

IIB—Functional Activity of the Cells Obtained

After stimulation, natural gamma 9 delta 2 T cells produce cytokines like TNF (“tumor necrosis factor”) and are cytotoxic to a number of cancer cells. In particular, gamma 9 delta 2 T cells have been shown to specifically lyse the Daudi (myeloma) cell line and not the RAJI cell line.

The functionality of the cells produced in the inventive cell culture method was tested in terms of two parameters: cytotoxic activity on a renal carcinoma tumor cell line (line 786-0, ATCC, reference CRL-1932) and a myeloma cell line (RAJI cells serving as negative control).

The cytotoxicity activity of the cells obtained by the method (cell density maintenance test at 0.2, 0.5, 1 million cells per ml, see hereinabove) at day 23, against these three cell lines, is given in Table 9.

It can be seen that the cells obtained by the method were indeed cytotoxic to the renal carcinoma and Daudi cell lines and, as expected, showed no significant cytotoxicity to the RAJI cell line. It was also shown that the cells were cytotoxic to the renal carcinoma cell line 786-0.

### Example III

### Proliferation Study on Frozen MNC Cells

An especially practical way of carrying out the method would be to be able to start with frozen cells. In fact, a cytapheresis can provide from 2 to 4 billion cells which it would be worthwhile to aliquot and freeze so as to perform several cultures from the same cytapheresis.

However, freezing can markedly alter cell viability and capacity to grow after thawing. The three MNC samples from the experiments in example II were frozen in 10% DMSO/4% HSA. A new expansion has been performed from frozen material (same protocol as in example II). The results of this expansion are given in Table 10.

It is seen that frozen cells can also generate large number of cells of very high purity.

The functional activity of fresh versus frozen cells was also tested in parallel on cells obtained by starting with fresh cells and with frozen cells. Two tests were carried out: the cytotoxicity test and the TNF release test.

The cytotoxicity test results on both fresh and frozen cells at day 21 are given in Table 11.

The TNF release test results on fresh and frozen cells at day 21 are given in Table 12.

The results of these function tests show that cells derived from frozen cells were not significantly different than cells derived from fresh cells. Different freezing media might improve the cell yield.

### Example IV

### Formulation of Cells for an Injectable Preparation

In view of injection in humans, the FCS must be eliminated and the cells taken up in a pharmaceutically acceptable buffer. Medium containing 4% HSA was tested. A new expansion was carried out using a new frozen cytapheresis. Six freezing conditions were evaluated:

Two freezing media: 10% DMSO in 4% HSA, 7.5% DMSO in FCS.

Both media were tested with three cell concentrations : 25, 50, 150 million cells per ml.

The expansion protocol was the same as in example II, except that cells were maintained at 0.5 million cells per ml starting from day 7.

The results of the expansion are given in Table 13.

There was little difference between these different freezing conditions, with slightly better results for the 7.5% DMSO/FCS freezing medium.

The cell preparation produced from the condition of 150 million cells per ml in FCS, 7.5% DMSO was formulated in 4% HSA.

The volume of the compositions was reduced with a “CytoMate®”, and the cells were then conditioned in 4% human serum albumin. To this end, the pellet was suspended in 100 to 200 ml of 4% human serum albumin, so as to obtain a cell suspension with a concentration comprised between 10 and 100 million cells per ml. Cells were counted and cell viability was measured, and the cells were then stored in a bag at 5° C.±3° C., so as to test the stability of the preparation.

The formulated cell product was tested for viability (trypan blue counting in a malassez cell) at time points 2 h, 4 h, 8 h and 22 h after formulation. The cell product contained more than 80% of viable cells until at least 22 h after formulation.

The formulated cell product was tested for functionality by the TNF release test at different time points to evaluate its stability.

The results are shown in Table 14.

It can be seen that the formulated cells were still capable of producing TNF after BrHPP stimulation, even 22 h after formulation. Furthermore, there was no significant difference in TNF production until 8 h after formulation.

TABLE 1

| | Certifi- | | | | |
|---|---|---|---|---|---|
| Company | cation | cGMP | FDA | Medium | References |
| Bio Media | ISO 9001 | BPF | E-DMF | Prolifix S3 | PROLIS3 |
| | ISO 9002 | | | | 20123431 |
| | EN 46002 | | | Prolifix S6 | PROLIS6 |
| | | | | | 20123456 |
| | | | | Ampicells | AMPICLX3 |
| | | | | X3 | 20123698 |
| Bio | | + | + | X-VIVO 10 | US04-380Q |
| Whittaker | | | | | |
| (Gibco) | | | | X-VIVO 15 | US04-418Q |
| (Life Tech- | ISO 9001 | | + | AIM V | 087.0112D |
| nologies) | | | | Pharma | K |
| Invitrogen | | | | grade | |
| Sigma | − | | − | Medium I | G-0916 |
| | | | | Medium II | G-0791 |
| Stem Cell | | + | | Stem Span | 09700 |
| | | | | H 2000 | |

TABLE 2

| | | | Surface | | |
|---|---|---|---|---|---|
| | | | area | Min. | Max. |
| | Supplier | Ref. | (cm²) | volume | volume |
| Lifecell PL732 | Nexell- | R4R2111 | 180 | 50 ml | 150 ml |
| 300 ml culture | Baxter | | | | |
| bags | | | | | |
| Evam | Stedim | FR0501S | 200 | / | / |
| 500 ml Nutribags | | TD | | | |
| 250 ml bags | CellGenix | 2P-0255 | 262 | / | / |
| (Vue Life 255 | | | | | |
| Culture Bags) | | | | | |
| NUNC 600 ml | Poly | 056968 | 185 | / | / |
| culture flasks, | | | | | |
| straight neck | | | | | |

TABLE 3

| | | | Final |
|---|---|---|---|
| | Supplier | Reference | dilution |
| hu-CD3-FITC/hu-CD56-PE | Immunotech | IM2075 | 1/5 |
| hu-Vδ2-FITC | Immunotech | IM1464 | 1/5 |
| hu-CD3-PE | Immunotech | IM1282 | 1/5 |
| hu-CD69-PC5 | Immunotech | IM2656 | 1/10 |
| Mouse IgG1, k-FITC | BD | 33814X | 1/10 |
| Mouse IgG1, k-R-PE | BD | 33815X | 1/10 |
| Mouse IgG1, k-CyC | Pharmingen | 71148L | 1/10 |

TABLE 4

| | | | Theoretical seeding | | |
|---|---|---|---|---|---|
| | Surface | | density (in proportion | | Actual number |
| Culture vessel | area | | to 24-well plates) | | of MNC seeded |
| Plates (24 wells) | 1.9 | cm² | 1. | 10⁶ | / |
| Lifecell 300 ml | 180 | cm² | 90. | 10⁶ | 100. 10⁶ |
| culture bags | | | | | |
| (Nexell) | | | | | |
| Evam 500 ml | 200 | cm² | 100. | 10⁶ | |
| Nutribags | | | | | |
| (Stedim) | | | | | |
| 250 ml bags Vue | 262 | cm² | 140. | 10⁶ | |
| Life (Cell Genix) | | | | | |
| NUNC 600 ml | 185 | cm² | 100. | 10⁶ | |
| culture flasks | | | | | |

TABLE 5
Study of PROLIFERATION in differents media in 24-well plates

| | | % Vd2 IN THE CULTURE | | | | TOTAL LYMPHOS (millions) | | | | TOTAL Vd2 (millions) | | |
|---|---|---|---|---|---|---|---|---|---|---|---|---|
| | FC- | SECURED | X-VIVO | X-VIVO | FC- | SECURED | X-VIVO | X-VIVO | FC- | SECURED | X-VIVO | X-VIVO |
| | I-* | HS | 10 | 15 | I-* | HS | 10 | 15 | I-* | HS | 10 | 15 |
| Day 10 | | | | | | | | | | | | |
| Donor 1 | 76.22 | 78.06 | 16.38 | 14.34 | 5.8 | 4.76 | 0.56 | 0.64 | 4.42 | 3.72 | 0.09 | 0.09 |
| Donor 2 | 83.82 | 80.87 | 30.56 | 34.83 | 8 | 5.2 | 0.64 | 1.2 | 6.71 | 4.21 | 0.20 | 0.42 |
| Donor 3 | 48.74 | 25.61 | 3.34 | 5.4 | 5 | 5.44 | 0.4 | 0.6 | 2.44 | 1.39 | 0.01 | 0.03 |
| Day 15 | | | | | | | | | | | | |
| Donor 1 | 80.63 | 60.05 | 23.17 | 17.89 | 8.4 | 4.6 | 0.4 | 0.7 | 6.77 | 2.76 | 0.09 | 0.13 |
| Donor 2 | 87.17 | 80.75 | 52.2 | 45.08 | 22.4 | 6.21 | 0.66 | 1.54 | 19.53 | 5.01 | 0.34 | 0.69 |
| Donor 3 | 62.37 | 13.93 | 3.95 | 6.32 | 7.14 | 13.2 | 0.1 | 0.3 | 4.45 | 1.84 | 0.00 | 0.02 |
| Day 22 | | | | | | | | | | | | |
| Donor 1 | 82.62 | 40.11 | 29.14 | 23.19 | 8.88 | 6.4 | 0.26 | 0.782 | 7.34 | 2.57 | 0.08 | 0.18 |
| Donor 2 | 84.35 | 77.67 | 76.53 | 69.51 | 27.36 | 9.8 | 1.3 | 4.76 | 23.08 | 7.61 | 0.99 | 3.31 |
| Donor 3 | 75.82 | 6.62 | NT | NT | 22.4 | 12 | <1 | <1 | 16.98 | 0.79 | <1 | <1 |
| Day 31 | | | | | | | | | | | | |
| Donor 1 | 82.39 | NT | NT | NT | 17.8 | <1 | <1 | <1 | 14.67 | <1 | <1 | <1 |
| Donor 2 | 92.62 | NT | NT | NT | 86.4 | <1 | <1 | <1 | 80.02 | <1 | <1 | <1 |
| Donor 3 | 73.25 | NT | NT | NT | 78.84 | <1 | <1 | <1 | 57.75 | <1 | <1 | <1 |

NT: not tested

TABLE 6
Expansion of gamma delta T cells from different sources
(triplicate experiments from 3 healthy donors)

| | D62 | | | D70 | | | D89 | | |
|---|---|---|---|---|---|---|---|---|---|
| | I | II | III | I | II | III | I | II | III |
| | % Vd2 in CULTURE | | | | | | | | |
| D 10 | | | | | | | | | |
| PBMC | 64.84 | 71.45 | 56.33 | 92.68 | 93.7 | 93.75 | 91.59 | 90.36 | 90.49 |
| MNC-FICOLI | 25.91 | 59.06 | 23.72 | 91.39 | 90.33 | 90.7 | 73.01 | 72.42 | 70.21 |
| MNC | 71.09 | 47.11 | 58.49 | 95.18 | 92.74 | 92.74 | 82.24 | 81.98 | 81.19 |
| D 14 | | | | | | | | | |
| PBMC | 65.09 | 76.2 | 54.73 | 93.25 | 98.02 | 97.8 | 91.5 | 94.04 | 92.52 |
| MNC-FICOLI | 3.84 | 60.17 | 9.27 | 95.83 | 95.58 | 95.6 | 71.81 | 58.93 | 54.63 |
| MNC | 76.83 | 45.56 | 62.48 | 97.25 | 96.83 | 96.72 | 81.31 | 78.1 | 76.55 |
| D 19 | | | | | | | | | |
| PBMC | 86.1 | 77.79 | 68.91 | 98.31 | 98.31 | 98.31 | 92.72 | 93.02 | 92.47 |
| MNC-FICOLI | 1.08 | 59.27 | 3.27 | 96.22 | 96.51 | 96.55 | 73.78 | 14.71 | 18.47 |
| MNC | 78.69 | 39.61 | 69.06 | 97.08 | 97.59 | 96.38 | 80.66 | 75.75 | 73.19 |
| | SPECIFIC Vd2 (millions) | | | | | | | | |
| D 10 | | | | | | | | | |
| PBMC | 2.59 | 2.29 | 1.8026 | 7.044 | 5.25 | 6.375 | 6.59 | 9.04 | 5.4294 |
| MNC-FICOL | 0.3 | 2.13 | 0.3795 | 6.946 | 6.14 | 6.1676 | 0.93 | 1.16 | 0.9829 |
| MNC | 1.68 | 0.9 | 1.8717 | 8.757 | 8.16 | 9.274 | 1.81 | 4.92 | 2.2733 |
| D 14 | | | | | | | | | |
| PBMC | 6.51 | 12.2 | 6.7865 | 22.38 | 27.4 | 31.296 | 16.1 | 16.9 | 18.504 |
| MNC-FICOL | 0.05 | 4.04 | 0.191 | 25.3 | 30.6 | 25.2384 | 1.9 | 1.27 | 3.2778 |
| MNC | 5.84 | 2.19 | 4.7485 | 37.34 | 33.3 | 34.04544 | 2.54 | 8.04 | 7.3488 |
| D 19 | | | | | | | | | |
| PBMC | 23.8 | 32.8 | 17.779 | 61.54 | 66.2 | 74.32236 | 51.2 | 40.3 | 48.824 |
| MNC-FICOL | 0.02 | 7.11 | 0.0523 | 91.41 | 85.5 | 72.9918 | 1.66 | 0.06 | 1.4776 |
| MNC | 11.1 | 2.14 | 13.052 | 90.19 | 85.4 | 69.77912 | 4.92 | 10 | 17.858 |

TABLE 7

| | | | | TOTAL LYMPHOS | | | SPECIFIC Vd2 | | |
|---|---|---|---|---|---|---|---|---|---|
| | % Vd2 in CULTURE | | | (millions) | | | (millions) | | |
| | D100 | D119 | D127 | D100 | D119 | D127 | D100 | D119 | D127 |
| D 10 | | | | | | | | | |
| FLASK | 64.89 | 79.05 | 27.91 | 160.5 | 263.52 | 266.22 | 104 | 208 | 74 |
| STEDIM | 72.81 | 79.81 | 61.49 | 174 | 307.47 | 340.2 | 127 | 245 | 209 |
| CELLGENIX | 79.97 | 85.25 | 58.89 | 257.87 | 451.52 | 363 | 206 | 385 | 214 |
| NEXELL | 80.14 | 84.52 | 73.06 | 262.26 | 398.75 | 382.11 | 210 | 337 | 279 |
| D 14 | | | | | | | | | |
| FLASK | 84.94 | 90.84 | 90.27 | 274.99 | 431.68 | 313.5 | 234 | 421 | 308 |
| STEDIM | 83.78 | 51.62 | 81.3 | 281.88 | 385.9 | 340.3 | 236 | 199 | 277 |
| CELLGENIX | 87.86 | 84.63 | 1.57 | 405 | 513.04 | 408 | 356 | 434 | 6 |
| NEXELL | 35.64 | 57.62 | 31.85 | 420 | 502.35 | 439.9 | 150 | 289 | 140 |
| D 20 | | | | | | | | | |
| FLASK | 71 | 5.8 | 0.57 | 430 | 533.4 | 456 | 305 | 31 | 3 |
| STEDIM | 81.38 | 31.11 | 3.85 | 351 | 511.7 | 420 | 286 | 159 | 16 |
| CELLGENIX | 75.13 | 78.43 | 7.01 | 580.8 | 699.2 | 591.6 | 436 | 548 | 41 |
| NEXELL | 84.14 | 78.83 | 20.62 | 607.20 | 741.00 | 396.50 | 511 | 584 | 82 |

TABLE 8

| | | | | TOTAL LYMPHOS | | | SPECIFIC Vd2 | | |
|---|---|---|---|---|---|---|---|---|---|
| | % Vd2 in CULTURE | | | (millions) | | | (millions) | | |
| | D 623 | D 762 | D 711 | D 623 | D 762 | D 711 | D 623 | D 762 | D 711 |
| D 0 | | | | | | | | | |
| initial | 2.17 | 3.61 | 1.01 | 100 | 100 | 100 | 2.17 | 3.61 | 1.01 |
| D 10 | | | | | | | | | |
| (triplicates) | | | | | | | | | |
| I | 91.28 | 92.05 | 89.45 | 541 | 586 | 615 | 494 | 539 | 550 |
| II | 91.48 | 91.76 | 89.43 | 552 | 562 | 604 | 505 | 516 | 540 |
| III | 91.85 | 91.79 | 90.11 | 579 | 576 | 625 | 532 | 557 | 609 |

| | D 623 | D 762 | D 711 | D 623 | D 762 | D 711 | D 623 | D 762 | D 711 |
|---|---|---|---|---|---|---|---|---|---|
| D 15 | | | | Billions | | | Billions | | |
| 0.2 million/m | 97.75 | 95.55 | 95.1 | 2.869 | 1.525 | 2.127 | 2.80 | 1.46 | 2.02 |
| 0.5 million/m | 97.54 | 97.09 | 96.21 | 3.047 | 2.341 | 2.759 | 2.97 | 2.27 | 2.65 |
| 1.5 million/m | 96.87 | 95.96 | 95.57 | 1.552 | 1.376 | 1.576 | 1.50 | 1.32 | 1.51 |
| D 21 | | | | | | | | | |
| 0.2 million/m | 97.8 | 94 | 94.2 | 7.699 | 5.761 | 5.553 | 7.53 | 5.42 | 5.23 |
| 0.5 million/m | 98.23 | 94.69 | 95.88 | 13.43 | 11.16 | 11.76 | 13.19 | 10.56 | 11.28 |
| 1.5 million/m | 97.79 | 96.52 | 96.67 | 4.926 | 3.879 | 4.92 | 4.82 | 3.74 | 4.76 |

TABLE 9
Cytotoxicity as % specific lysis on 3 cell lines

| | Donor | | | | | | | | |
|---|---|---|---|---|---|---|---|---|---|
| | D 711 | D 711 | D 711 | D 762 | D 762 | D 762 | D623 | D623 | D623 |
| | Maintenance concentratio [symbol] | | | | | | | | |
| Target | at 0.2 | at 0.5 | at 1.5 | at 0.2 | at 0.5 | at 1.5 | at 0.2 | at 0.5 | at 1.5 |
| 786-0 | | | | | | | | | |
| RATIO 0.3/1 | 15.93 | 15.8 | 4.12 | 16.2 | 17.3 | 10.09 | 4.28 | 5.618 | 4.692 |
| RATIO 3/1 | 42.98 | 32.7 | 32.7 | 48.6 | 61 | 54.02 | 33.5 | 18.17 | 18.63 |
| RATIO 30/1 | 65.38 | 54 | 34.4 | 83.1 | 68 | 65.25 | 46.7 | 61.04 | 41.7 |
| RAJI | | | | | | | | | |
| RATIO 0.3/1 | 6.826 | 13.3 | 10.6 | 7.81 | 3.92 | 8.814 | 5.73 | 3.863 | 13.71 |
| RATIO 3/1 | 55.64 | 8.28 | 5.73 | 14.4 | 19.1 | 11.69 | 6.32 | 6.943 | 10.59 |
| RATIO 30/1 | 15.32 | 14.1 | 11.3 | 15.6 | 13.2 | 16.83 | 14.2 | 16.88 | 16.88 |
| DAUDI | | | | | | | | | |
| RATIO 0.3/1 | 43.87 | 49.9 | 47.3 | 54.8 | 59.5 | 49.97 | 29.1 | 46.52 | 53.09 |
| RATIO 3/1 | 59.51 | 54.7 | 55.5 | 64.6 | 67.1 | 58.97 | 56.1 | 61.27 | 58.63 |
| RATIO 30/1 | 60.39 | 56.1 | 53.7 | 68.9 | 60.1 | 60.02 | 60.3 | 58.29 | 62.42 |

TABLE 10

| | | | | TOTAL LYMPHOS | | | SPECIFIC Vd2 | | |
|---|---|---|---|---|---|---|---|---|---|
| | % Vd2 in CULTURE | | | (billions) | | | (billions) | | |
| | D 623 | D 762 | D 711 | D 623 | D 762 | D 711 | D 623 | D 762 | D 711 |
| D 0 | 2.17 | 3.61 | 1.01 | 0.100 | 0.100 | 0.100 | 0.0022 | 0.0036 | 0.0010 |
| D 11 | 94.82 | 92.4 | 92.13 | 0.623 | 0.609 | 0.579 | 0.590 | 0.563 | 0.533 |
| D 17 | 97.27 | 88.02 | 94.18 | 1.196 | 0.657 | 1.125 | 1.1633 | 0.5783 | 1.0595 |

TABLE 11
Cytotoxicity of cells obtained from fresh or frozen MNC on mRCC cell line

| | | | | | | | Positive | Negative |
|---|---|---|---|---|---|---|---|---|
| | D 711 | D 711 | D 762 | D 762 | D623 | D623 | control | control |
| 786-0 | fresh | frozen | fresh | frozen | fresh | frozen | G12 | A4, 5 |
| RATIO 0.3/1 | 5.06495 | 4.6827 | 5.256 | 11.671 | 7.2152 | 9.85516 | 0.7645214 | 1.1945647 |
| RATIO 3/1 | 21.765 | 26.257 | 25.25 | 24.011 | 29.183 | 33.2089 | 6.163954 | 3.0103031 |
| RATIO 30/1 | 49.9806 | 59.513 | 73.59 | 86.033 | 52.812 | 57.5541 | 38.512767 | 7.7407795 |

TABLE 12
TNF alpha production (pg/ml) on 25,000 cells

| | D711 | | D762 | | D623 | |
|---|---|---|---|---|---|---|
| | fresh | frozen | fresh | frozen | fresh | frozen |
| BrHPP | 903.3 | 1336.16 | 782.08 | 973.7 | 1332.7 | 1442.36 |
| stimulation | | | | | | |
| Without | <20 | <20 | <20 | <20 | <20 | <20 |
| BrHPP | | | | | | |

TABLE 13

| | % Vd2 in culture | | | | | | TOTAL LYMPHOS (billions) | | | | | |
|---|---|---|---|---|---|---|---|---|---|---|---|---|
| Freezing | 1 | 2 | 3 | 4 | 5 | 6 | 1 | 2 | 3 | 4 | 5 | 6 |
| condition | 150 | 50 | 25 | 150 | 50 | 25 | 150 | 50 | 25 | 150 | 50 | 25 |
| Day | HSA | HSA | HSA | FC-I- | FC-I- | FC-I- | HSA | HSA | HSA | FC-I- | FC-I- | FC-I- |
| 0 | 6.7 | 7.57 | 7.07 | 7.07 | 6.92 | 7.05 | 0.1 | 0.1 | 0.1 | 0.1 | 0.1 | 0.1 |
| 7 | 92.88 | 94.12 | 93.88 | 91.87 | 94.41 | 93.02 | 0.5 | 0.52 | 0.49 | 0.55 | 0.48 | 0.51 |
| 10 | 97.75 | 98.1 | 98.3 | 98.17 | 98.11 | 98.4 | 2.66 | 2.37 | 2.43 | 2.58 | 2.4 | 2.85 |
| 17 | 97.88 | 98.44 | 98.4 | 98.21 | 98.45 | 98.7 | 14.2 | 15.36 | 12.6 | 20.6 | 12.1 | 25.85 |

| | TOTAL Vd2 (billions) | | | | | | |
|---|---|---|---|---|---|---|---|
| Freezing | 1 | 2 | 3 | 4 | 5 | 6 | |
| condition | 150 | 50 | 25 | 150 | 50 | 25 | |
| Day | HSA | HSA | HSA | FC-I- | FC-I- | FC-I- | D |
| 0 | 0.007 | 0.008 | 0.007 | 0.007 | 0.007 | 0.007 | 0 |
| 7 | 0.471 | 0.489 | 0.460 | 0.505 | 0.453 | 0.474 | 7 |
| 10 | 2.6 | 2.3 | 2.4 | 2.5 | 2.4 | 2.8 | 10 |
| 17 | 13.9 | 15.1 | 12.4 | 20.2 | 11.9 | 25.5 | 17 |

TABLE 14
Pg/ml of alpha TNF produced by 25 000 cells

| | Triplicate means | | | |
|---|---|---|---|---|
| | Time | | | |
| | T = 2 h | T = 4 h | T = 8 h | T = 22 h |
| Stimulation | HSA 4% | HSA 4% | HSA 4% | HSA 4% |
| With BrHPP | 381.358 | 359.624 | 327.31 | 127.56 |
| No BrHPP | <20 | <20 | <20 | <20 |

1-20. (canceled)

21. A method for preparing a gamma delta (γδ) T lymphocyte composition comprising culturing a biological preparation comprising at least 50 million mononuclear cells in the presence of a synthetic activator compound of gamma delta T lymphocytes and a cytokine.

22. The method according to claim 21, wherein the biological preparation is a blood, plasma or serum sample.

23. The method according to claim 22, wherein the biological preparation is from a cytapheresis.

24. The method according to claim 21, wherein the biological preparation comprises more than 10×10⁷ cells.

25. The method according to claim 21, wherein the biological preparation has previously been frozen.

26. The method according to claim 21, further comprising maintaining the cells at a density less than about 5×10⁶ cells/ml during said culturing step.

27. The method according to claim 21, wherein the cells are cultured for a time period greater than or equal to about 10 days.

28. The method according to claim 27, wherein said cells are cultured between 10 and 25 days.

29. The method according to claim 21, wherein the synthetic activator compound of gamma delta T lymphocytes is a ligand of the T cell receptor of said gamma delta T lymphocytes.

30. The method according to claim 29, wherein the synthetic activator compound of said gamma delta T lymphocytes is selected from the group consisting of phosphohalohydrin compounds, phosphoepoxide compounds and bisphosphonate compounds.

31. The method according to claim 30, wherein the synthetic activator compound of said gamma delta T lymphocytes is selected in the group consisting of the following compounds:
3-(bromomethyl)-3-butanol-1-yl-diphosphate (BrHPP);
3-(iodomethyl)-3-butanol-1-yl-diphosphate (IHPP);
3-(chloromethyl)-3-butanol-1-yl-diphosphate (ClHPP);
3-(bromomethyl)-3-butanol-1-yl-triphosphate (BrHPPP);
3-(iodomethyl)-3-butanol-1-yl-triphosphate (IHPPP);
α,γ-di-[3-(bromomethyl)-3-butanol-1-yl]-triphosphate (diBrHTP);
α,γ-di-[3-(iodomethyl)-3-butanol-1-yl]-triphosphate (diIHTP);
3,4,-epoxy-3-methyl-1-butyl-diphosphate (Epox-PP);
3,4,-epoxy-3-methyl-1-butyl-triphosphate (Epox-PPP); and
α,γ-di-3,4,-epoxy-3-methyl-1-butyl-triphosphate (di-Epox-TP).

32. The method according to claim 21, wherein the cytokine is selected in the group consisting of interleukin-2 and interleukin-15.

33. The method according to claim 21, wherein the cytokine is used at a concentration between about 150 U/ml and about 500 U/ml.

34. The method according to claim 21, wherein said method produces a composition of gamma delta T lymphocytes having the following characteristics:
said composition comprises more than 80% gamma delta T cells, and
said composition comprises more than 100 million viable and functional gamma delta T cells.

35. A method for enriching the population of functional gamma delta T lymphocytes in a biological sample comprising culturing cells from a cytapheresis in the presence of a synthetic activator compound of gamma delta T lymphocytes.

36. The method according to claim 35, further comprising the addition of a cytokine selected from the group consisting of interleukin-2 and interleukin-15.

37. The method according to claim 35, wherein said cells are cultured under conditions that ensure that cell density is maintained at essentially below 5×10⁶ cells/ml.

38. The method according to claim 36, wherein said cytokine is added one to 72 hours after the culturing of said cells is initiated.

39. The method according to claim 36, wherein said cytokine is added to said culture of cells at the time culturing of said cells is initiated.

40. The method according to claim 35, further comprising the step of recovering some or all of said gamma delta T lymphocytes.

41. The method according to claim 40, further comprising formulating said recovered gamma delta T lymphocytes into a pharmaceutically acceptable composition.

42. The method according to claim 38, further comprising the step of recovering some or all of said gamma delta T lymphocytes.

43. The method according to claim 42, further comprising formulating said recovered gamma delta T lymphocytes into a pharmaceutically acceptable composition.

44. The method according to claim 39, further comprising the step of recovering some or all of said gamma delta T lymphocytes.

45. The method according to claim 44, further comprising formulating said recovered gamma delta T lymphocytes into a pharmaceutically acceptable composition.

46. A composition comprising a population of cells comprising more than 80% functional gamma delta T lymphocytes and comprising more than 100 million gamma delta T lymphocytes and a carrier or excipient.

47. The composition according to claim 46, further comprising human serum albumin.

48. The composition according to claim 46, further comprising a cytokine selected from the group consisting of IL-2 and IL-15.

49. A method of stimulating the immune defenses of a subject comprising the administration of a composition comprising a population of cells composed of more than 80% functional gamma delta T lymphocytes, more than 100 million gamma delta T lymphocytes and a carrier or excipient to said subject.

50. The method according to claim 49, wherein said method treats an infectious disease, a parasitic disease, or cancers.
